(19) United States
(12) Patent Application Publication
(10) Pub. No.: US 2013/0338653 A1
(43) Pub. Date: Dec. 19, 2013

(54) MINIMALLY INVASIVE APOCRINE GLAND INCAPACITATION APPARATUS AND METHODS

(71) Applicant: Paul Joseph Weber, Queenstown (NZ)

(72) Inventor: Paul Joseph Weber, Queenstown (NZ)

(21) Appl. No.: 13/661,335

(22) Filed: Oct. 26, 2012

Related U.S. Application Data
(60) Provisional application No. 61/659,300, filed on Jun. 13, 2012.

(51) Int. Cl. A61B 18/14; A61B 18/20; A61B 18/04; A61B 18/18

(52) U.S. Cl. CPC A61B 18/14; A61B 18/1815; A61B 18/203; A61B 18/04
USPC 606/13; 606/45; 606/33; 606/27

(57) ABSTRACT

Methods, apparatus and systems for modifying apocrine glands are disclosed herein. A method for modifying apocrine glands may comprise inserting a tissue dissecting and modifying wand (TDM) into an incision in a patient's skin. The TDM may comprise a tip having a plurality of protrusions with lysing segments positioned between the protrusions. The TDM may also comprise an energy window positioned on top of the TDM that is configured to deliver energy to incapacitate apocrine glands. After separating tissue using the lysing segment(s) to define a target region, the energy window may be activated and moved around within the target region to incapacitate apocrine glands. In some implementations, the energy window may be activated prior to separating the tissue such that the tissue is separated while apocrine glands are incapacitated within the target region.

### RELATED APPLICATIONS

This application claims the benefit under 35 U.S.C. §119(e) of U.S. Provisional Patent Application No. 61/659,300 named inventor Paul J. Weber, filed Jun. 13, 2012 and titled “MINIMALLY INVASIVE APOCRINE GLAND MODIFICATION APPARATUS AND METHODS,” which application is incorporated herein by reference in its entirety.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1a is a perspective view of an embodiment of a tissue dissector and modifier with an energy window on the upper side of the device.

FIG. 1b is a side elevation view of the embodiment previously depicted in FIG. 1a.

FIG. 1c is a front elevation view of the embodiment previously depicted in FIG. 1a.

FIG. 1d is a front elevation view illustrating the protrusions and lysing segment of an alternative embodiment of a tissue dissector and modifier wherein the lysing segment connecting the two protrusions is centered substantially midway between the upper and lower portions of the protrusions.

FIG. 1e is a front elevation view illustrating the protrusions and lysing segment of an alternative embodiment of a tissue dissector and modifier, wherein the lysing segment connecting the two protrusions is positioned above the midline between the upper and lower portions of the protrusions.

FIG. 1f is a front elevation view illustrating the protrusions and lysing segment of an alternative embodiment of a tissue dissector and modifier, wherein the lysing segment connecting the two protrusions is positioned below the midline between the upper and lower portions of the protrusions.

FIG. 1g is a cross-sectional view of an embodiment illustrating some examples of some of the canals that may be used with the device.

FIG. 2a is a perspective view of an embodiment of a tissue dissector and modifier with a thermochromic-based energy window on the upper side of the device.

FIG. 2b is a side elevation view of the embodiment previously depicted in FIG. 2a.

FIG. 2c is a front elevation view of some thermochromic-based energy window components of an embodiment previously depicted in FIG. 2a.

FIG. 3a is a perspective view of an embodiment of a tissue dissector and modifier with a target-tissue-impedance-matched-microwave-based energy window on the upper side of the device.

FIG. 3b is a side elevation view of the embodiment previously depicted in FIG. 3a.

FIG. 3c is a front elevation view of some target-tissue-impedance-matched-microwave-based energy window components of an embodiment previously depicted in FIG. 3a.

FIG. 4a is a perspective view of an embodiment of a tissue dissector and modifier without an energy window.

FIG. 4b is a side elevation view of the embodiment previously depicted in FIG. 4a.

FIG. 5 is a flow chart illustrating one implementation of a method for incapacitating apocrine glands.

### DETAILED DESCRIPTION

In humans apocrine glands are only found in the armpits, and the areas around the nipples, genitals and anus. Apocrine glands are commonly thought to be associated with various human odors. However, actual apocrine gland secretions are initially an odorless, milky, viscous secretion; only after the action of bacteria may the secretions then emit an odor. Interestingly, even the term apocrine is a misnomer as the glands are merocrine in nature. Animal and human skin is usually composed of at least three layers including: (1) the outermost surface epidermis which contains pigment cells and pores; (2) the dermis or leather layer; and (3) the subdermis which is usually fat, fibrous tissue or muscle. Portions of the apocrine gland may reside in the lower dermis and subdermis. Apocrine sweat glands are coiled and tubular yet larger than eccrine sweat glands; apocrine glands also have different mechanism of secretion. Apocrine glands contain myoepithelial cells (from Greek myo-, “muscle”), specialized epithelial cells located between the gland cells and the underlying basal lamina. Myoepithelial cell contractions squeeze the gland and discharge the accumulated secretions. Apocrine sweat glands discharge in the canals of hair follicles. The secretory activities of the gland cells and the contractions of myoepithelial cells are controlled by both the autonomic nervous system and by the circulating hormones. The secretions patterns of the milky protein-rich fluid may be transient, intermittent, or variable. Apocrine glands may begin secreting at puberty; apocrine secretions may contain pheromones, chemicals that communicate information to other individuals by altering their hormonal balance. The degree of secretory activity may be regulated by both neural and hormonal mechanisms. (Source: Wikipedia)

It is noteworthy that the coiled base of the apocrine glands often protrudes into the relatively soft subcutaneous tissue below the dermis; sympathetic nerve endings innervate apocrine glands. At least these two features of the gland may make the apocrine gland susceptible to trauma or energy application in the lower dermis and upper subcutaneous tissues.

In some cases, apocrine glands may be overly productive or the result of their secretions abnormally pungent. This may result in an undesirable odor which may be embarrassing and/or otherwise psychologically stressful. As such, in certain areas, it may be desirable to incapacitate some or all of the apocrine glands in such areas. Incapacitation of apocrine glands may encompass any method that prevents the glands from continuing to function in their normal capacity by secreting perspiration. This may be accomplished, for example, by cutting/lysing the gland and/or its nervous tissue supply, burning or otherwise introducing thermal or other energetic trauma to the gland and/or its nervous tissue supply, introduction of collagen deposits or scarring to the glands and/or the surrounding tissue, or removing the glands.

Various implementations of methods are disclosed herein for incapacitating apocrine glands. Such methods may be performed using a Tissue Dissecting and Modifying Wand (“TDM”). Examples of various embodiments of such wands may be found in U.S. Pat. No. 6,203,540 titled “Ultrasound and Laser Face-Lift and Bulbous Lysing Device,” U.S. Pat. No. 6,391,023 titled “Thermal Radiation Facelift Device,” U.S. Pat. No. 6,432,101 titled “Surgical Device for Performing Face-Lifting Using Electromagnetic Radiation,” U.S. Pat. No. 6,440,121 titled “Surgical Device For Performing Face-Lifting Surgery Using Radiofrequency Energy,” U.S. Pat. No. 6,974,450 titled “Face-Lifting Device,” and U.S. Pat. No. 7,494,488 titled “Facial Tissue Strengthening and Tightening Device and Methods.” The “Detailed Description of the Invention” section of each of these patents is hereby incorporated herein by specific reference. With respect to U.S. Pat. No. 6,203,540 titled “Ultrasound and Laser Face-Lift and Bulbous Lysing Device,” the section titled “Description of the Preferred Embodiments” is hereby incorporated herein by specific reference.

Although the TDM has been described in these cited patents for use in connection with face lift procedures, it has recently been discovered that this tool may also be useful in certain apocrine gland procedures, as disclosed herein. Apocrine glands may be susceptible to various forms of trauma including: direct lysis or cutting up of the coil or duct, thermal or other energetic effects on the cells of the coil or duct (which may be direct or indirect), or denervation by traumatizing or interrupting the sympathetic nervous supply to the apocrine gland. A fourth mechanism may be at play following trauma around the apocrine gland and may be associated with post traumatic collagen deposition or scarring. Thermal damage to collagen is likely brought about by hydrolysis of cross-linked collagen molecules and reformation of hydrogen bonds resulting in loss of portions or all of the characteristic collagen triple-helix. New collagen formed as the result of trauma and some diseases is technically scar tissue. The encroachment of post traumatically derived collagen may influence already traumatized apocrine glands.

Because the methods for incapacitating apocrine glands are typically performed in a patient's underarm region, the temperatures to which the tissue is heated may be higher than temperatures that would typically be involved in facial rejuvenation procedures. For example, it may be the case that facial tissue is heated to temperatures that are lower than what would be most useful in apocrine gland incapacitation procedures. In some implementations, apocrine glands may be incapacitated by heating the tissue to a temperature of about 72-80° C.

The TDM may dissect a plane in the upper subcutaneous tissue. It is possible that the cutting segments alone may traumatize or lyse portions of the apocrine gland that may extend about 2 mm into the upper subcutaneous fat. It may therefore be desirable to provide a device that can access these glands from underneath the upper subcutaneous fat. It is also possible that when electrically energized with electro-cutting current, the TDM may possess a plasma field that may traumatize apocrine glands in a potentially lethal fashion. The TDM may be “energized” by various forms of energy in its top side energy window, as described in greater detail below. Such energy absorptions may result in the resultant formation of heat which may, in turn, damage apocrine glands themselves, or their surrounding environment or their nerve supply in order to fully or partially incapacitate the apocrine glands.

In some embodiments, energy may be delivered from one or more energy windows so as to heat tissue to a temperature of about 72° C. to about 80° C. Various methods may therefore be implemented in which the amount of energy and/or the delivery time may be adjusted so as to heat the tissue to within a desired temperature range. Temperature sensors may therefore be incorporated on or near the energy windows to allow a surgeon to heat the tissue to a desired temperature or within a desired temperature range. In some embodiments, the sensor may be configured to provide an average temperature over a particular period of time and or over a particular range of distances within the tissue. Systems consistent with the disclosure provided herein may be configured to prevent or to shut down or otherwise limit energy transfer if a particular tissue temperature were beyond a threshold or alternatively if an average temperature threshold is reached.

Temperature sensors that may be useful in connection with embodiments disclosed herein include, but are not limited to, resistance temperature sensors, such as carbon resistors, film thermometers, wire-wound thermometers, or coil elements. Some embodiments may comprise thermocouples, pyrometers, or non-contact temperature sensors, such as total radiation or photoelectric sensors. In some embodiments, one or more temperature sensors may be coupled with a processor and/or a monitor to allow a surgeon to better visualize or otherwise control the delivery of energy to selected areas for apocrine gland incapacitation. For example, some embodiments may be configured such that a surgeon can visualize the temperature of tissue positioned adjacent to one or more locations along the TDM to ensure that such temperatures are within a desired temperature range. Some embodiments may alternatively, or additionally, be configured such that one or more temperature sensors are coupled with a processor in a feedback loop such that energy delivery may be automatically adjusted by the system in response to temperature data. For example, when temperatures exceed a particular threshold, such as somewhere between about 65° C. and about 90° C., the system may be configured to shut down or otherwise limit further energy delivery. In some such embodiments, the threshold may be between about 68° C. and about 75° C.

Some embodiments may comprise a feedback means, such as a visual, audible, or tactile feedback means, to notify the surgeon when the temperature has reached a particular threshold and/or the TDM has been positioned in a particular location within the target region for a particular time period. The feedback means may be configured with multiple thresholds with different feedback at each threshold. For example, at a first threshold, the TDM may be configured to deliver a first noise and at a second threshold the TDM may be configured to deliver a second noise. The second noise may be louder than the first noise to indicate a greater urgency for changing the energy delivery and/or moving the TDM from its current location under the patient's skin.

In many implementations of methods according to the present disclosure, the TDM may be used to incapacitate apocrine glands located in or near a patient's underarm region. Some facial or neck rejuvenation procedures using the TDM are done by delivering energies of about 20 J/cm². By contrast, in certain preferred implementations of methods for incapacitating apocrine glands using the TDM, a higher energy delivery may be employed than would be with a facial or neck rejuvenation procedure. For example, some implementations for incapacitating apocrine glands may be performed by delivering energy at a level 20% or more than 20 J/cm².

In some implementations, all or substantially all of the apocrine glands may be incapacitated.

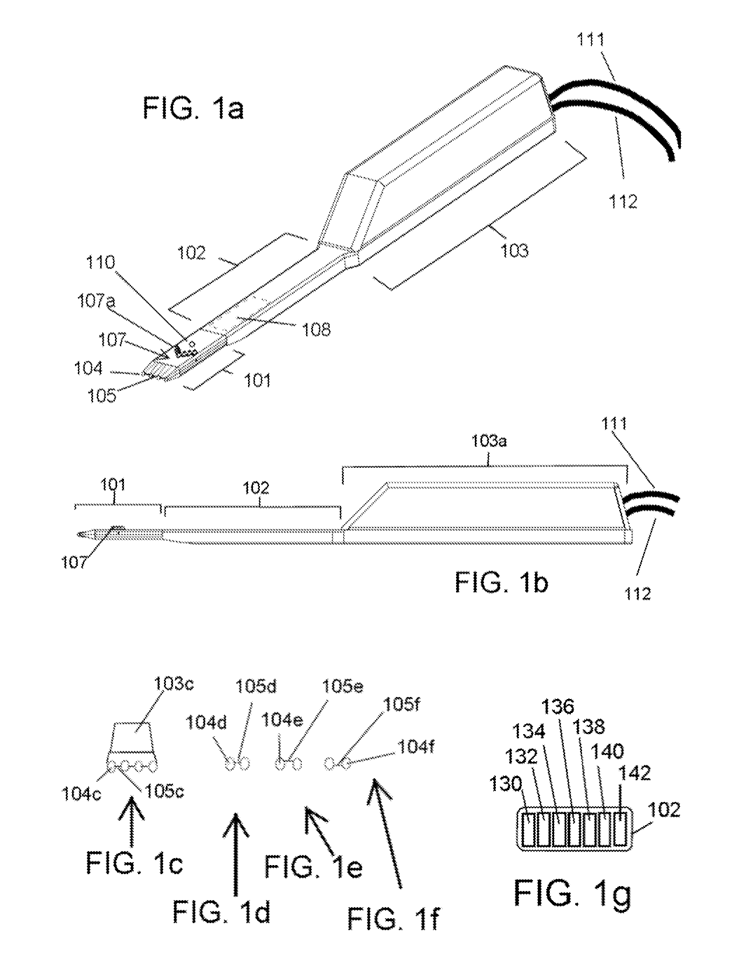

Further details regarding various embodiments will now be provided with reference to the drawings. FIG. 1a is a perspective view of an embodiment of a TDM with an electrosurgically energized energy window 107 on the upper side of the device. It should be noted that the term “energy window” is intended to encompass what is referred to as a planar-tissue-altering-window/zone in U.S. Pat. No. 7,494,488 and, as described later, need not be electrosurgically energized in all embodiments. The tip shown in this embodiment has four relative protrusions and three relative recessions and provides for a monopolar tip conductive element.

The tip 101 may be slightly larger than the shaft 102, which leads to handle 103. Electro-coagulation and electro-cutting energy arrives in leads 111 & 112 and may travel by wiring through the handle and shaft to termini 107a, which are part of energy window 107. Electro-cutting and electro-coagulation currents may be controlled outside the TDM at an electrosurgical generator, such as the Bovie Aaron 1250™ or Bovie Icon GP™. In an embodiment, the tip may measure about 1 cm in width and about 1-2 mm in thickness. Sizes of about one-fifth to about five times these dimensions may also have possible uses. In some embodiments, the tip can be a separate piece that is secured to shaft by a variety of methods such as a snap mechanism, mating grooves, plastic sonic welding, etc. Alternatively, in some other embodiments, the tip can be integral or a continuation of shaft made of similar metal or materials. In some embodiments, the tip may also be constructed of materials that are both electrically non-conductive and of low thermal conductivity; such materials might comprise, for example, porcelain, ceramics, glass-ceramics, plastics, varieties of polytetrafluoroethylene, carbon, graphite, and graphite-fiberglass composites.

In some embodiments, the tip may be constructed of a support matrix of an insulating material (e.g., ceramic or glass material such as alumina, zirconia). External power control bundles 111 & 112 connect to electrically conductive elements to bring RF electrosurgical energy from an electrosurgical generator down the shaft 102 to electrically conductive lysing elements 105 mounted in the recessions in between the protrusions 104. In some embodiments, the protrusions may comprise bulbous protrusions. In the depicted embodiment, the tip 101 may alternatively be made partially or completely of concentrically laminated or annealed-in wafer layers of materials that may include plastics, silicon, glass, glass/ceramics, cermets or ceramics. Lysing elements 105 may also be made partially or completely of a cermet material. Alternatively, in a further embodiment the tip may be constructed of insulation covered metals or electroconductive materials. In some embodiments, the shaft may be flat, rectangular or geometric in cross-section or substantially flattened. In some embodiments, smoothing of the edges of the shaft may reduce friction on the skin surrounding the entrance wound. In some further embodiments, the shaft may be made of metal or plastic or other material with a completely occupied or hollow interior that can contain insulated wires, electrical conductors, fluid/gas pumping or suctioning conduits, fiber-optics, or insulation.

In some embodiments, shaft plastics, such as polytetrafluoroethylene may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, graphite-fiberglass composites. Depending upon the intended uses for the device, an electrically conductive element internal to shaft may be provided to conduct electrical impulses or RF signals from an external power/control unit (such as a Valleylab™ electrosurgical generator) to another energy window 108. In some embodiments, energy windows 107 and/or 108 may only be relatively planar, or may take on other cross-sectional shapes that may correspond with a portion of the shape of the shaft, such as arced, stair-step, or other geometric shapes/curvatures. In the embodiments depicted in FIGS. 1a & 1b, energy window 107 is adjacent to protrusions 104, however other embodiments are contemplated in which an energy window may be positioned elsewhere on the shaft 102 or tip 101 of the wand, and still be considered adjacent to protrusions 104. For example, in an embodiment lacking energy window 107, but still comprising energy window 108, energy window 108 would still be considered adjacent to protrusion 104. However, if an energy window was placed on handle 103, such an energy window would not be considered adjacent to the protrusions 104.

The conduit may also contain electrical control wires to aid in device operation. Partially hidden from direct view in FIGS. 1a & 1b, and located in the grooves defined by protrusions 104 are electrically conductive tissue lysing elements 105, which, when powered by an electrosurgical generator, effects lysing of tissue planes on forward motion of the device. The lysing segments may be located at the termini of conductive elements. In some embodiments, one or more sensors may be positioned on or near location 110. Other embodiments may comprise one or more sensors on any other suitable location on the TDM, including but not limited to on the protrusions or otherwise on the tip, and on the shaft. Sensors that may be useful include thermal sensors, photoelectric or photo optic sensors, cameras, etc. In some embodiments, one or more sensors may be used to monitor the local post passage electrical impedance or thermal conditions that may exist near the distal tip of the shaft or on the tip. Some embodiments may also comprise one or more sensors incorporating MEMS (Micro Electro-Mechanical Systems) technology, such as MEMS gyroscopes, accelerometers, and the like. Such sensors may be positioned at any number of locations on the TDM, including within the handle in some embodiments.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermistors, and may feed to analog amplifiers which, in turn, feed analog digital converters leading to a microprocessor. In some embodiments, internal or external ultrasound measurements may also provide information which may be incorporated into a feedback circuit. In an embodiment, an optional mid and low frequency ultrasound transducer may also be activated to transmit energy to the tip and provide additional heating and may additionally improve lysing. In some embodiments, a flashing visible light source, for example, an LED, can be mounted on the tip may show through the upper skin flap to identify the location of the device.

Some embodiments may comprise a low cost, disposable, and one-time-use device. However, in some embodiments intended for multiple uses, the tip's electrically conductive tissue lysing elements be protected or coated with materials that include, but are not limited to, Silverglide™ non-stick surgical coating, platinum, palladium, gold and rhodium. Varying the amount of protective coating allows for embodiments of varying potential for obsolescence capable of either prolonging or shortening instrument life.

In some embodiments, the electrically conductive lysing element portion of the tip may arise from a plane or plate of varying shapes derived from the aforementioned materials by methods known in the manufacturing art, including but not limited to cutting, stamping, pouring, molding, filing and sanding. In some embodiments, the electrically conductive lysing element 105 may comprise an insert attached to a conductive element in the shaft or continuous with a formed conductive element coursing all or part of the shaft. In some embodiments, an electrically conductive element or wiring 111 brings RF electrosurgical energy down the shaft to electrically conductive lysing elements 105 associated in part with the recessions. In an embodiment, the electrosurgical energy via 111 is predominately electro-cutting.

In some embodiments, the electrically conductive element or wiring may be bifurcated to employ hand switching if an optional finger switch is located on handle. The electrically conductive element or wiring leading from the shaft into the handle may be bundled with other leads or energy delivering cables, wiring and the like and may exit the proximal handle as insulated general wiring to various generators (including electrosurgical), central processing units, lasers and other sources as have been described herein. In some embodiments, the plate making up lysing segments 105 may be sharpened or scalloped or made to slightly extend outwardly from the tip recessions into which the plate will fit.

Alternatively, in some embodiments, since cutting or electrical current may cause an effect at a distance without direct contact, the lysing element may be recessed into the relative recessions or grooves defined by the protrusions 104 or, alternatively, may be flush with protrusions 104. In some further adjustable embodiments, locations of the electrically conductive lysing elements with respect to the protrusions may be adjusted by diminutive screws or ratchets. The plate, which in some embodiments is between 0.01 mm and 1 mm thick, can be sharpened to varying degrees on its forward facing surface. It is possible that plate sharpness may increase the efficiency with which electricity will pass from the edge cutting the target tissue. Sometimes, however, proper function even when variably dull or unsharpened may be unhampered since electrosurgical cutting current may cut beyond the electroconductive edge by a distance of over 1 mm.

In some embodiments, the electrically conductive lysing element may also exist in the shape of a simple wire of 0.01 mm to 3 mm. In some embodiments, the wire may measure between 0.1 mm and 1 mm. Such a wire may be singly or doubly insulated as was described for the plate and may have the same electrical continuities as was discussed for the planar (plate) version. In some embodiments, an electrosurgical current for the electrically conductive lysing element is of the monopolar “cutting” variety and setting and may be delivered to the tip lysing conductor in a continuous fashion or, alternatively, a pulsed fashion. The surgeon can control the presence of current by a foot pedal control of the electrosurgical generator or by button control on the shaft (forward facing button). The amount of cutting current may be modified by standard interfaces or dials on the electro surgical generator. In some embodiments, the electrosurgically energized tip current can be further pulsed at varying rates by interpolating gating circuitry at some point external to the electrosurgical generator by standard mechanisms known in the art, preferably at rates of about 1 per second to about 60 per second. For some embodiments, the electrically conductive lysing element is a monopolar tip in contact with conductive elements in the shaft leading to external surgical cable leading to an electrosurgical generator from which emanates a grounding or dispersive plate which may be placed elsewhere in contact with the patient's skin, such as the thigh.

Such circuitry may be controlled and gated/wired from the cutting current delivery system of the electro surgical generator. Acceptable electrosurgical generators may include Valley Lab Force 1 B™ with maximum P-P voltage of 2400 on “cut” with a rated load of 300 Ohms and a maximum power of 200 Watts, 35 maximum P-P voltage of 5000 on “coagulate” with a rated load of 300 Ohms, and a maximum power of 75 Watts ValleyLab Force 4 has a maximum P-P voltage of 2500 on “cut” with a rated load of 300 Ohms and a maximum power of 300 Watts, 750 kHz sinusoidal waveform output, maximum P-P voltage of 9000 on “coagulate” with a rated load of 300 Ohms and a maximum power of 120 Watts using a 750 kHz damped sinusoidal with a repetition frequency of 31 kHz. In an embodiment, the tip may also be manufactured from multilayer wafer substrates comprised of bonded conductive strips and ceramics. Suitable conductive materials include but are not limited to those already described for tip manufacture.

In alternative embodiments, the electrically conductive lysing elements may be bifurcated or divided into even numbers at the relative recessions, insulated and energized by wiring to an even number of leads in a bipolar fashion and connected to the bipolar outlets of the aforementioned electrosurgical generators. Rings partly or completely encircling the shaft of the hand unit can be linked to a partner bipolar electrode at the tip or on the energy window. Such bipolar versions may decrease the available power necessary to electrically modify certain tissues, especially thicker tissues.

FIG. 1b is a side elevation view of the embodiment previously depicted in FIG. 1a. In the depicted embodiment, tip 101 may be made of materials that are both electrically non-conductive and of low thermal conductivity such as porcelain, epoxies, ceramics, glass-ceramics, plastics, or varieties of polytetrafluoroethylene. Alternatively, the tip may be made from metals or electroconductive materials that are completely or partially insulated. Note the relative protrusions and relative recessions are not completely visible from this viewing angle. In some embodiments, the relative recessions of the tip is the electrically conductive tissue lysing element 105 (usually hidden from view at most angles) which may have any geometric shape including a thin cylindrical wire; the electrically conductive lysing element can be in the shape of a plate or plane or wire and made of any metal or alloy that does not melt under operating conditions or give off toxic residua. Optimal materials may include but are not limited to steel, nickel, alloys, palladium, gold, tungsten, copper, and platinum. Metals may become oxidized thus impeding electrical flow and function.

FIG. 1c is a front elevation view of an embodiment of the embodiment previously depicted in FIG. 1a. In this depicted embodiment, there are 4 protrusions and 3 lysing segment recessions 105c; the vertical height of a protrusion may be about 3 mm and the horizontal width may be about 2 mm. In this depicted embodiment, the relatively oval protrusions 104c may be shaped similarly to a commercial jetliner nose cone in order to reduce drag and lower resistance to facilitate tissue passage. In some embodiments, tip protrusion shapes may take on a wide variety of geometric shapes including, but not limited to, stacked rectangles or tapered thin rectangles as discussed elsewhere. In some further embodiments the relative projection shapes that may include, but should not be limited to: spheroid, sphere, sphere on cylinder, sphere on pyramid, sphere on cone, cone, cylinder, pyramid, and polyhedron.

FIG. 1d is a front elevation view of an alternative embodiment having two protrusions 104d and one lysing segment (recession) wherein the lysing segment 105d connecting the two protrusions is substantially centered midway between the upper and lower portions of the protrusions. In the depicted embodiment, the vertical height of the protrusions may be about 3 mm and the horizontal width may be about 2 mm. Thus, the lysing segment may be placed about 1.5 mm from the upper portion of the protrusion. If the upper portion of the protrusion is run close to the bottom of the dermis then the lysing segment will lyse approximately 1.5 mm from the lowermost portion of the relatively rigid dermis if the plane of dissection is made adjacent to the subdermal subcutaneous fat. The closer to the lower dermis that the energized lysing segment passes there may be more potential to denature certain skin structures which may be glands.

FIG. 1e is a front elevation view of another embodiment having two protrusions and one lysing segment 105e wherein the lysing segment connecting the two protrusions 104e is substantially centered in the upper third of the way (on the upper side) between the upper and lower portions of the protrusions. In the depicted embodiment, the vertical height of the protrusions may be about 3 mm and the horizontal width may be about 2 mm. Thus, the lysing segment may be placed about 1 mm from the upper portion of the protrusion. If the upper portion of the protrusion is run close to the bottom of the dermis then the lysing segment will lyse approximately 1 mm from the lowermost portion of the relatively rigid dermis if the plane of dissection is made adjacent to the subdermal subcutaneous fat. This embodiment places the lysing segment 33% closer to the lower dermis than the embodiment in FIG. 1d with even more potential to denature certain skin structures, including glands.

FIG. 1f is a front elevation view of another embodiment having two protrusions and one lysing segment wherein the lysing segment 105f connecting the two protrusions 104f is substantially centered in the lower third (on the lower side) between the upper and lower portions of the protrusions. In the depicted embodiment, the vertical height of the protrusions may be about 3 mm and the horizontal width may be about 2 mm. Thus, the lysing segment may be placed about 2 mm from the upper portion of the protrusion. If the upper portion of the protrusion is run close to the bottom of the dermis then the lysing segment will lyse approximately 2 mm from the lowermost portion of the relatively rigid dermis if the plane of dissection is made adjacent to the subdermal subcutaneous fat. This embodiment places the lysing segment 33% farther from the lower dermis than the embodiment in FIG. 1d with less potential to denature certain skin structures, including glands.

As discussed above, some embodiments may be configured such that the position of the lysing segment(s) relative to the protrusions is adjustable, such as adjustable between the embodiments shown in FIGS. 1d-1f.

FIG. 1g is a cross-sectional view of an embodiment of a TDM illustrating some examples of some of the canals that may be used with the device. For example, canal 130 may comprise an electrode canal for delivering electrical energy to one or more of the lysing segments and/or the energy window(s). Canal 132 may comprise an optics canal for delivering and/or receiving optical signals or energy, such as a LASER, fiber optics, intense pulse light, or for receiving an optical sensor. Canal 134 may comprise a vacuum tube for sucking fluids away from the surgical site, such as bodily fluids and/or fluids introduced by the TDM during the surgery. One or more of these canals may be configured for delivering one or more fluids using the TDM. For example, canal 136 may comprise a fluid delivery canal for delivering an ionic fluid, such as a saline solution. Canal 136 may be configured to deliver a fluid that is both ionic and an anesthetic, such as a tumescent anesthesia. In some embodiments, canal 136 may be configured to deliver a fluid containing multiple individual fluids, such as a Klein Formula. Canal 138 may serve as a coaxial cable canal, such as for delivering a microwave signal to the energy window, for example. Canals 140 and 142 may comprise duplicates of any one of the foregoing canals 130-138. One or more of the canals 130-142 may be coated with copper or another conductive metal to insulate the signals from those within other canals. It should be understood that although these canals are not depicted in other figures, any of the embodiments described herein may include one or more such canals. It should also be understood that although the canals shown in FIG. 1g are shown as having rectangular cross sections, any other cross sectional shape, including but not limited to circular cross sections, may be used.

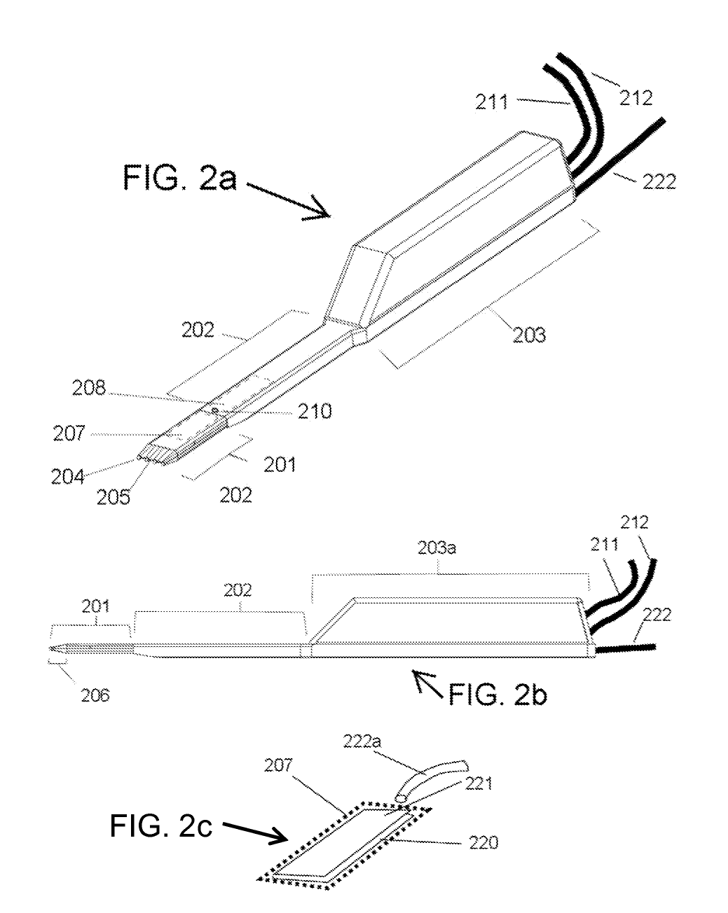

FIG. 2a is a perspective view of an embodiment of a TDM with an alternative energy window 207 on the upper side of the device configured to hold a thermochromic film. It should be noted that the term “energy window” is intended to encompass what is referred to as a planar-tissue-altering-window/zone in U.S. Pat. No. 7,494,488 and, as described herein, need not contain a thermochromic film in all embodiments. Additionally, the “energy window” may comprise a variety of other energy emitting devices, including radiofrequency, microwave, intense pulsed light, LASER, thermal, and ultrasonic. Certain components of the energy window, such as the electro-conductive components of the energy window, could comprise a cermet.

The tip 201 may be slightly larger than the shaft 202, which leads to handle 203. Electrosurgical energy may be delivered in leads 211 and 212 whereas LASER energy may be delivered by fiberoptic 222 or a waveguide and may travel by fiberoptic or waveguide through the handle and shaft to energy window 207, which may comprise a thermochromic film. A second energy window 208 may also be included in some embodiments, and may comprise yet another thermochromic film or another variety of energy emitting device. Electro-cutting and electro-coagulation currents may be controlled outside the TDM at an electrosurgical generator, such as the Bovie Aaron 1250™ or Bovie Icon GP™. In some embodiments, the tip may measure about 1 cm in width and about 1-2 mm in thickness. Sizes of about one-fifth to about five times these dimensions may also have possible uses. In some embodiments, the tip can be a separate piece that may be secured to a shaft by a variety of methods, such as a snap mechanism, mating grooves, plastic sonic welding, etc. Alternatively, in some other embodiments, the tip can be integral or a continuation of a shaft made of similar metal(s) or material(s). In some embodiments, the tip may also be constructed of materials that are both electrically non-conductive and of low thermal conductivity; such materials might comprise, for example, porcelain, ceramics, glass-ceramics, plastics, varieties of polytetrafluoroethylene, carbon, graphite, and graphite-fiberglass composites.

In some embodiments, the tip may be constructed of a support matrix of an insulating material (e.g., ceramic or glass material such as alumina, zirconia). External power control bundles 211 may connect to electrically conductive elements to bring RF electrosurgical energy from an electrosurgical generator down the shaft 202 to electrically conductive lysing elements 205 mounted in the recessions in between protrusions 204. In some embodiments, the protrusions may comprise bulbous protrusions. In the depicted embodiment, the tip 201 may alternatively be made partially or completely of concentrically laminated or annealed-in wafer layers of materials that may include plastics, silicon, glass, glass/ceramics or ceramics. Alternatively, in a further embodiment, the tip may be constructed of insulation covered metals or electroconductive materials. In some embodiments, the shaft may be flat, rectangular, or geometric in cross-section, or may be substantially flattened. In some embodiments, smoothing of the edges of the shaft may reduce friction on the skin surrounding the entrance wound. In some further embodiments, the shaft may be made of metal or plastic or other material with a completely occupied or hollow interior that can contain insulated wires, electrical conductors, fluid/gas pumping or suctioning conduits, fiber-optics, or insulation.

In some embodiments, shaft plastics, such as polytetrafluoroethylene, may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, and/or graphite-fiberglass composites. Depending upon the intended uses for the device, an electrically conductive element internal to the shaft may be provided to conduct electrical impulses or RF signals from an external power/control unit (such as a Valleylab™ electrosurgical generator) to another energy window 208. In some embodiments, energy windows 207 and/or 208 may only be relatively planar, or may take on other cross-sectional shapes that may correspond with a portion of the shape of the shaft, such as arced, stair-step, or other geometric shapes/curvatures. In some embodiments, energy window 208 may comprise another thermochromic film. In the embodiments depicted in FIGS. 2a & 2b, energy window 207 is adjacent to protrusions 204, however other embodiments are contemplated in which an energy window may be positioned elsewhere on the shaft 202 or tip 201 of the wand, and still be considered adjacent to protrusions 204. For example, in an embodiment lacking energy window 207, but still comprising energy window 208, energy window 208 would still be considered adjacent to protrusion 204. However, if an energy window was placed on handle 203, such an energy window would not be considered adjacent to protrusions 204.

The conduit(s) may also contain electrical control wires to aid in device operation. Partially hidden from direct view in FIGS. 2a & 2b, and located in the recessions defined by protrusions 204, are electrically conductive tissue lysing elements 205, which, when powered by an electrosurgical generator, effects lysing of tissue planes on forward motion of the device. The lysing segments may be located at the termini of conductive elements. In some embodiments, optional locations for multiple impedance sensors or multiple thermal sensors include location 210, which may be used to monitor the local post passage electrical impedance or thermal conditions that may exist near the distal tip of the shaft.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermistors, and may feed to analog amplifiers which, in turn, feed analog digital converters leading to a microprocessor. In some embodiments, internal or external ultrasound measurements may also be taken during a procedure with the TDM.

FIG. 2b is a side elevation view of the embodiment previously depicted in FIG. 2a. In the depicted embodiment, tip 201 which terminates in protrusions 206 may be made of materials that are both electrically non-conductive and of low thermal conductivity such as porcelain, epoxies, ceramics, glass-ceramics, plastics, or varieties of polytetrafluoroethylene. Alternatively, the tip may be made from metals or electroconductive materials that are completely or partially insulated. Note the relative protrusions and relative recessions are not completely visible from this viewing angle. The tip shown in this embodiment has four relative protrusions and three relative recessions and provides for a monopolar tip conductive element. In some embodiments, the electrically conductive tissue lysing element(s) 205 (usually hidden from view at most angles), which may have any geometric shape including a thin cylindrical wire, may be positioned within the relative recessions of the tip. The electrically conductive lysing element can be in the shape of a plate or plane or wire and made of any metal or alloy that does not melt under operating conditions or give off toxic residua. Optimal materials may include but are not limited to steel, nickel, alloys, palladium, gold, tungsten, copper, and platinum. Metals may become oxidized thus impeding electrical flow and function.

Thus far in medicine and surgery, thermochromic films have principally seen use as sensors or detection devices and thus absorb energy and contribute to modifying said energy into quantifiable information or data; for example, applying organic thermochromic indicators to surgical instruments with radiofrequency “jaws” to visually indicate to a surgeon when a given temperature is reached, however such an “organically sensitive” device has replacement cartridges (e.g., as shown in U.S. Pat. No. 7,041,102 titled “Electrosurgical working end with replaceable cartridges,” which is hereby incorporated by reference).

Herein, the use of thermochromic films is presented for a diametrically opposite purpose: to pump a defined quantity of energy into a living system to alter tissue. As opposed to traditional electrical resistance based thermal emission, thermochromic films may have an extremely well defined capacity for digital regulation and thus may yield a more exact or controllable application of energy to target tissues. Organic and inorganic thermochromic materials tend to have a fast response time over a broad wavelength band and return to the transparent state when the LASER beam subsides. So, thermochromic materials may act more as a safety switch wherein, instead of having a separate sensor for temperature, a “fail-safe” mechanism would be to set the thermochromic to shut down transmission if, using round numbers only, for example, the temperature of the thermochromic film exceeded 100 degrees centigrade depending upon the speed at which the TDM was moving. Other embodiments are contemplated in which the temperature threshold for limiting energy transmission ranges from about 65 to 90° C. In some such embodiments, the threshold may be between 68 to 75° C. Vanadium Dioxide (VO₂) as a thermochromic film may see many potential uses, as it has such a rapid transition (in femtoseconds) between the crystalline lattices of the metallic and semiconductor phase transition geometries. Regarding industrial use, for example, at temperatures below 69 centigrade VO₂ is a transparent semiconductor, but at just a few degrees higher, VO₂ may display its usefulness as a “reflective window coating.” VO₂'s rapid phase transition may see usefulness in optical switches and even faster computer memory.

FIG. 2c depicts an embodiment of the thermochromic energy window embodiment previously depicted in FIG. 2a. This depicted embodiment includes energy window 207, which is configured to comprise all or a portion of a thermochromic media 220, which is, in turn, substantially covered by a covering layer 221. In some embodiments, fiber optic 222 carries LASER energy derived from a LASER generator, into and through the handle, down the shaft and into the thermochromic media. In some embodiments, a wave guide may carry the LASER energy down the shaft. In some embodiments, Vanadium Dioxide (VO₂) may be used as the inorganic thermochromic material and may be covered by a covering layer. In some embodiments, the Vanadium Dioxide layer is about 200-300 microns in thickness. In some embodiments, the Vanadium Dioxide layer ranges from about 10 microns to about 1000 microns. In some embodiments, the covering layer is silica. In some embodiments, the covering layer comprises a transparent dielectric, quartz, alumina, sapphire, diamond, and/or ceramic. In some further embodiments, plastics may serve as a covering layer. In some embodiments, an Nd:YAG (neodymium yttrium, aluminum, garnet) LASER may energize the thermochromic media. In some embodiments, a Candela™ Gentle YAG™1064 nm LASER is configured to energize a fiberoptic that thereupon leads into the TDM thermochromic window. In other embodiments, Manganese Strontium Oxide may serve as the thermochromic layer. In some embodiments, diode LASERS may be used to energize the thermochromic material. In some embodiments, metal vapor LASERS and/or semiconductor-based LASERS may be used to energize the thermochromic material. Metal vapor LASERS may include, but are not limited to, copper vapor and gold vapor. The power source may be more helpful if it runs continuously but is not too strongly absorbed to get the thermochromic effect when VO₂ changes in reflectivity.

Near-infrared LASERS may have some advantages over visible range LASERS in that contrast may be enhanced. In some embodiments, fiberoptics may carry the LASER energy. In some embodiments, a wave guide carries the LASER energy to the thermochromic film. In some embodiments, the thermochromic film may be configured to measure about 2×1 cm in area. In some embodiments, the thermochromic film may be configured to deliver about 40 J/cm². In some embodiments, about 1 J/cm² to about 200 J/cm² may be delivered.

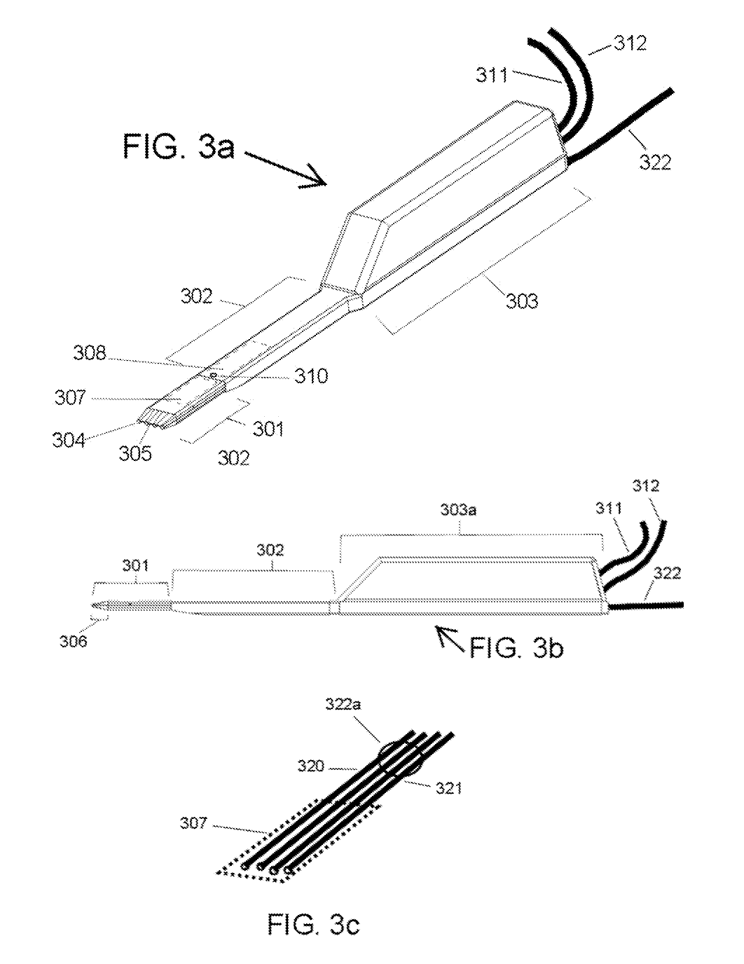

FIG. 3a is a perspective view of an embodiment of a tissue dissector and modifier with a target-tissue-impedance-matched-microwave-based energy window on the upper side of the device. A target-tissue-impedance-matched-microwave emission system (TTIMMES) may be advantageous over previously available microwave based medical treatment systems because it is difficult to model tissue against water because the dielectric associated with water differs from that of blood, which differs from that of tissue, and so on, especially after coagulum formation. Both non-impedance-matched-microwave and radiofrequency treatments may suffer from this concern. Beneficially for microwaves there is limited coagulum formation, and deeper penetration of energy into the tissues. With impedance matching, energy is not reflected back from the tissues into the microwave emitting antennae as the energy proceeds uni-directionally through the coaxial cable and into the target tissue. A controllable solid state source (e.g., MicroBlate™) of a super-high frequency (SHF) microwave emission band of 14.5 GHz system that is impedance matched has been shown to produce a depth of penetration of about 1.6 mm using coaxial antennae measuring just 2.2 mm (Int'l Journal of Hyperthermia 28: 43-54, 2012).

FIG. 3a is a perspective view of an embodiment of a TDM with an alternative energy window 307 on the upper side of the device configured to hold an array of impedance-matched-microwave emitting antennae. It should be noted that the term “energy window” is intended to encompass what is referred to as a planar-tissue-altering-window/zone in U.S. Pat. No. 7,494,488 and, as described herein, need not contain a microwave emitter in all embodiments. Additionally, the “energy window” may comprise a variety of other energy emitting devices, including radiofrequency, thermochromic, intense pulsed light, LASER, thermal, and ultrasonic. It should also be understood that the term “energy window” does not necessarily imply that energy is delivered uniformly throughout the region comprising the energy window. Instead, some energy window implementations may comprise a series of termini or other regions within which energy is delivered with interspersed regions within which no energy, or less energy, is delivered. This configuration may be useful for some implementations to allow for alteration of certain tissue areas with interspersed areas within which tissue is not altered, or at least is less altered. This may have some advantages for certain applications due to the way in which such tissue heals.

The tip 301 may be slightly larger than the shaft 302, which leads to handle 303. Electrosurgical energy may be delivered in leads 311 and 312, whereas gigahertz microwave energy may be delivered by coaxial cable bundle 322 through the handle and shaft to energy window 307, which may comprise four antennae termini. Some embodiments comprise between 1 and 10 antennae. Some embodiments may comprise a flat microwave emitting device. A second energy window 308 may also be included in some embodiments, and may comprise yet another microwave emitter or another variety of energy emitting device. Electro-cutting and electro-coagulation currents may be controlled outside the TDM at an electrosurgical generator, such as the Bovie Aaron 1250™ or Bovie Icon GP™. In some embodiments, the tip may measure about 1 cm in width and about 1-2 mm in thickness. Sizes of about one-fifth to about five times these dimensions may also have possible uses.

In some embodiments, the tip can be a separate piece that may be secured to a shaft by a variety of methods, such as a snap mechanism, mating grooves, plastic sonic welding, etc. Alternatively, in some other embodiments, the tip can be integral or a continuation of a shaft made of similar metal(s) or material(s). In some embodiments, the tip may also be constructed of materials that are both electrically non-conductive and of low thermal conductivity; such materials might comprise, for example, porcelain, ceramics, glass-ceramics, plastics, varieties of polytetrafluoroethylene, carbon, graphite, and graphite-fiberglass composites.

In some embodiments, the tip may be constructed of a support matrix of an insulating material (e.g., ceramic or glass material such as alumina, zirconia). External power control bundles 311 may connect to electrically conductive elements to bring RF electrosurgical energy from an electrosurgical generator down the shaft 302 to electrically conductive lysing elements 305 mounted in the recessions in between protrusions 304. In some embodiments, the protrusions may comprise bulbous protrusions. In the depicted embodiment, the tip 301 may alternatively be made partially or completely of concentrically laminated or annealed-in wafer layers of materials that may include plastics, silicon, glass, glass/ceramics or ceramics. Alternatively, in a further embodiment, the tip may be constructed of insulation covered metals or electroconductive materials. In some embodiments, the shaft may be flat, rectangular, or geometric in cross-section, or may be substantially flattened. In some embodiments, smoothing of the edges of the shaft may reduce friction on the skin surrounding the entrance wound. In some further embodiments, the shaft may be made of metal or plastic or other material with a completely occupied or hollow interior that can contain insulated wires, electrical conductors, fluid/gas pumping or suctioning conduits, fiber-optics, or insulation.

In some embodiments, shaft plastics, such as polytetrafluoroethylene may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, graphite-fiberglass composites. Depending upon the intended uses for the device, an electrically conductive element internal to shaft may be provided to conduct electrical impulses or RF signals from an external power/control unit (such as a Valleylab™ electrosurgical generator) to another energy window 308. In some embodiments, energy windows 307 and/or 308 may only be relatively planar, or may take on other cross-sectional shapes that may correspond with a portion of the shape of the shaft, such as arced, stair-step, or other geometric shapes/curvatures. In some embodiments, energy window 308 may comprise another microwave emitter.

The conduit(s) may also contain electrical control wires to aid in device operation. Partially hidden from direct view in FIGS. 3a & 3b, and located in the recessions defined by protrusions 304, are electrically conductive tissue lysing elements 305, which, when powered by an electrosurgical generator, effects lysing of tissue planes on forward motion of the device. The lysing segments may be located at the termini of conductive elements.

In some embodiments, one or more impedance sensors and/or thermal sensors may also be provided, such as at location 310 for example, which may be used to monitor the local post passage electrical impedance or thermal conditions that may exist near the distal tip of the shaft.

FIG. 3c depicts an embodiment of the target-tissue-impedance-matched-microwave-based emission system (TTIMMES) previously depicted in FIG. 3a. This depicted embodiment includes energy window 307, which is configured to comprise a bundle of microwave antennae 322a further comprising singular antennae, such as 320 and 321. Coaxial cable bundle 322 carries gigahertz microwave energy derived from a super high frequency (SHF) generator, into and through the handle, down the shaft and into the coaxial antennae. In some embodiments, a flat microwave emitter may be placed in energy window 307. In some embodiments, flat microwave emission devices are comprised of a “microstrip” in which an antenna is printed on a circuitboard. In some embodiments, the circuitboard may be coated with polytetrafluoroethylene, and may be seated on an alumina substrate.

In some embodiments, a controllable solid state source (N5183A MXG Microwave Analog Signal Generator from Agilent Technologies™) of a super-high frequency (SHF) microwave emission band of 36 GHz system that is impedance matched drives the coaxial cables to emit microwaves.

Some embodiments of the energy window may also comprise one or more LASERs that may also be used through the fiberoptic and may be controlled at the electromagnetic energy source by a footswitch. In some embodiments, the planar tissue-altering-window/zone may be an optical window that allows laser light to exit the shaft and irradiate nearby target tissue. A light delivery means, which can be a hollow waveguide or single or multiple optical fibers (such as metal coated plastic manufactured by Polymicro Technologies™, Inc. of Phoenix, Ariz.) may be contained in an external conduit. The external conduit may comprise, for example an articulating arm as is commonly used in surgical laser systems. Additional control wires and power may be delivered to the handpiece via the external conduit. However, using foot-pedal control from an electromagnetic energy radiation source or control interface, dial, or panel will likely be less cumbersome for the surgeon and reduce the expense of handpiece finger-control manufacture.

Some embodiments may use an energy window comprising Germanium, which may allow for egress of laser light and collection of data by thermal sensors, and such energy window may be of varying size. In another embodiment, a multiplicity of optical fibers may terminate at specific or random places within the energy window. Such bare or coated fiberoptic termini may protrude from, be flush with, or be recessed into, other materials comprising the energy window. Such bare or coated fiberoptic termini may protrude from, be flush with or be recessed into other materials comprising the energy window.

Bare fiberoptics comprising ethylene oxide sterilizable may be seated in a thermally nonconductive background, preferably at uniform 90 degree angles, but variable angles between 0 and 180 degrees may also be efficacious. The preferred light delivery means may depend on the wavelength of the laser used. Infrared light emitted by the heated tissue can also be collected through the window and sensed by an infrared detector to measure the tissue temperature. For C0₂ laser irradiance, reliable sources include standard operating room units, such as the Encore Ultrapulse® from Lumenis Corp. of Santa Clara, Calif., which is capable of providing continuous C0₂ laser energy outputs of 2-22 mJoules at 1-60 Watts. Older models of the Coherent Ultrapulse™ may also be suitable (Coherent™ now owned by Lumenis™). The hollow section of shaft may act as a waveguide or may contain a metal-coated plastic fiberoptic or waveguide to allow laser light to pass through and exit from window near tip. The window allows egress for laser light delivered to apparatus. Lasers usable in various embodiments disclosed herein include both pulsed and continuous wave lasers, such as C0₂, erbium YAG, Nd:YAG and Yf:YAG. The beam diameter can be changed as desired, as those skilled in the art will appreciate. However, this list is not intended to be self-limiting and other wavelength lasers may be used. Some embodiments of the energy window may comprise an intense, pulsed, non-coherent, non-LASER, such as a filtered flashlamp that emits a broadband of visible light. The flashlamp, such as a smaller version of that used by ESC/Sharplan™, Norwood, Mass. (500-1200 nm emission range; 50 J/sqcm fluence; 4 ms pulse; 550 nm filter) may occupy the handle or window/zone of the embodiment. Should IPL flashlamp accommodations increase shaft thickness significantly, the 1 cm entrance incisions can be easily transformed into 1.5 cm incisions along the anatomic lines and combined with a perpendicular incision of 1-1.5 cm to form a small A to T flap from which a much larger diameter shaft can enter, yet be easy to sew.

The flashlamp may emit optical and thermal radiation that can directly exit the energy window, or may be reflected off a reflector to exit through the window. The reflector may have a parabolic shape to effectively collect radiation emitted away from the window, which may be made of a wide variety of glass that transmits optical, near infrared, and infrared light (e.g., quartz, fused silica and germanium.) Emission spectra can be filtered to achieve the desired effects. Thermal emissions or visible radiation absorption may locally heat the dermis to alter collagen. Thermal sensors may also be used to control or reduce overheating. In order to eliminate excessive heating of the shaft and the surrounding facial tissue, the flashlamp and reflector may be thermally isolated by low thermal conductivity materials or cold nitrogen gas that may be pumped through a hollow or recessed portion of the shaft and/or handle. The handle can be an alternative location for the flashlamp so that emitted radiation may be reflected by a mirror through the window/zone.

Direct piezoelectric versions of the energy window may impart vibrational energy to water molecules contained in target tissues passing adjacent to the piezo material(s). Temperature elevations may cause collagenous change and cell wall damage, however, ultrasonic energy application may have disruptive effects at the subcellular level as well. Crystals that acquire a charge when compressed, twisted or distorted are piezoelectric. Electrical oscillations applied to certain ceramic wafers may cause ultrasonic mechanical vibrations. Energy output for piezoelectric window/zones typically ranges from about 1-30 J, with a preferred range of about 1-6 J in a surgical device moving about 1 cm/second. As with all other embodiments, temperature and impedance sensors providing intraoperative real-time data can modulate energy input into the piezoelectric, which may be energized by one or more conductive elements in the shaft in further connection with the control unit and/or power supply. The energy window for a thermally energized embodiment may allow thermal energy to escape from within the shaft, and wherein the tip can be integral or a continuation of shaft made of similar metal or materials. The tip may also be constructed of materials that are both electrically non-conductive and of low thermal conductivity; such materials might be porcelain, ceramics or plastics. Portions of the tip and shaft may be covered with Teflon® to facilitate smooth movement. Teflon® may also be used to coat portions of an antenna, such as a microwave antenna, such that the energy is delivered in a more uniform fashion. Alternatively, the filament may be fixedly attached to the shaft. The hot filament may emit optical and thermal radiation that can directly exit the energy window or be reflected off a reflector to also exit through window. The reflector may have a parabolic shape to effectively collect all optical and thermal radiation emitted away from the window. The hot filament can be a tungsten carbide filament similar to those used in high power light bulbs. The wavelength may be adjusted and controlled by adjusting the filament temperature/current. The window can be selected from a wide variety of glass that transmits optical, near infrared and infrared light (e.g., quartz, fused silica and germanium.) The tissue penetration depth may depend on the wavelength of the light (e.g., 1 μm may penetrate through about 10 mm, 10 μm may penetrate through about 0.02 mm).

The broad emission spectrum from the hot filament may be filtered to achieve the desired tissue effect. In particular, filtering the emission spectrum to heat the dermis to temperatures of approximately 72 to 82° C. may cause the desired glandular incapacitation particularly for areas of the skin likely to be treated using methods disclosed herein such as the underarm area. It should be understood that this range of temperatures may be applicable to any of the other embodiments disclosed herein and is not limited solely to the filament embodiment. The optimum spectral filtering may depend on skin thickness and structure. Thermal sensors connected to the control unit by electrical wire may be used to monitor the temperature of tissue that is in contact with the shaft. In order to eliminate excessive heating of the shaft and the surrounding facial tissue, the heating element and/or reflector may be thermally isolated by low thermal conductivity materials. The heating element may be isolated by not touching the shaft, whereas the reflector can have an isolating layer where it attaches to the shaft. In addition, cold nitrogen gas may be injected through tube and pumped out through the hollow shaft to cool the tip and shaft.

Some embodiments may place the hot filament in the handle while emitted optical and thermal radiation is reflected off a mirror through the window. An alternative embodiment may allow for tissue heating to be achieved by direct contact with a hot surface where electric current flowing through wires heats a resistive load made of single or multiple elements to a user selected temperature. The resistive load could be a thin film resistor and the film temperature could be estimated from the measured resistance. Alternatively, separate thermal sensors placed close to the heating element may be used to measure temperatures, which may be sent to a control unit to control the current through the resistive load. Cold gas or liquid(s) can be injected through tubes and pumped out through the shaft. Also, the heating element could be the hot side of a Peltier thermoelectric cooler which advantageously cools the opposite surface below ambient temperature with differences of up to about 40° C. Thermal embodiments wherein heat is derived via magnetic or frictional methods may bring about similar tissue alterations.

It has been discovered that some embodiments may also be effective without means for and energy window. For example, in some embodiments lacking an energy window, energy delivered by or otherwise at the lysing elements may be sufficient to at least partially incapacitate the apocrine glands within a target region as the tissue is separated. In such embodiments and implementations it may be useful to provide a higher energy such a higher level of electrosurgical energy (for example current flow). In some embodiments and implementations, the energy at the lysing elements may be increased beyond what would otherwise be needed just to separate tissue into planes. Although in some embodiments, one may be able to incapacitate apocrine glands by using only the requisite energy needed to separate tissue In other embodiments, energy may be increased (such as an increase of 5% to 500%) to increase the efficacy of incapacitating apocrine glands without the use of an energy window. In other embodiments, energy may be increased (such as an increase of 5% to 150%) to increase the efficacy of incapacitating apocrine glands without the use of an energy window In other embodiments, energy may be increased (such as an increase of 10% to 30%) to increase the efficacy of incapacitating apocrine glands without the use of an energy window.

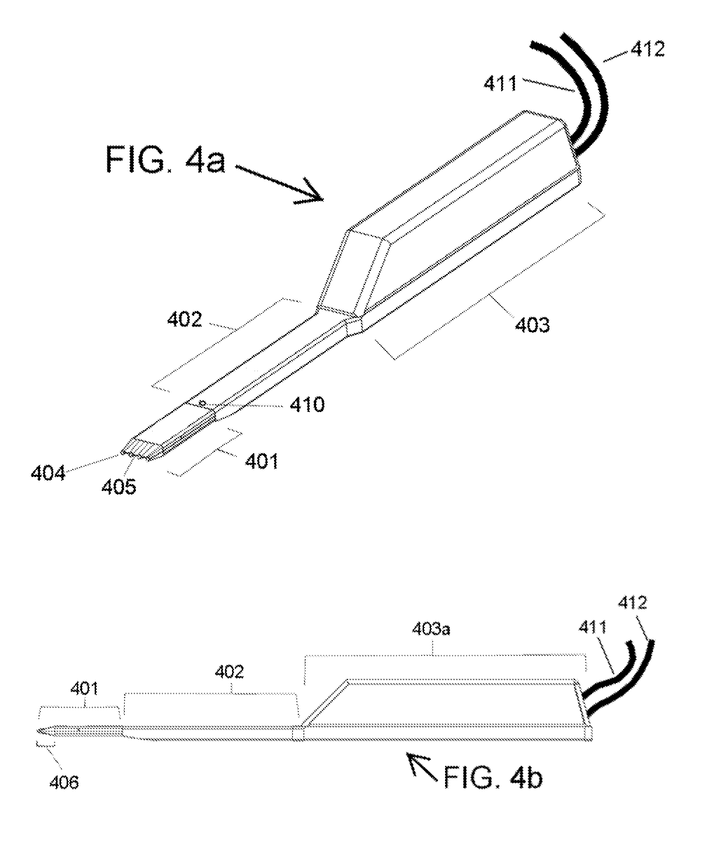

FIG. 4a is a perspective view of an embodiment of a TDM without an electrosurgically energized energy window. The tip 401 may be slightly larger than the shaft 402, which leads to handle 403. Electro-coagulation and electro-cutting energy arrives in leads 411 & 412 and may travel by wiring through the handle and shaft 402 to electrically conductive lysing elements 405 mounted in the recessions in between the protrusions 404. In the depicted embodiment, the tip 401 may alternatively be made partially or completely of concentrically laminated or annealed-in wafer layers of materials that may include plastics, silicon, glass, glass/ceramics or ceramics. Alternatively, in a further embodiment the tip may be constructed of insulation covered metals or electroconductive materials. In some embodiments, the shaft may be flat, rectangular or geometric in cross-section or substantially flattened. In some embodiments, smoothing of the edges of the shaft may reduce friction on the skin surrounding the entrance wound. In some further embodiments, the shaft may be made of metal or plastic or other material with a completely occupied or hollow interior that can contain insulated wires, electrical conductors, fluid/gas pumping or suctioning conduits, fiber-optics, or insulation.

In some embodiments, the tip may be constructed of a support matrix of an insulating material (e.g., ceramic or glass material such as alumina, zirconia).

In an embodiment, the tip may measure about 1 cm in width and about 1-2 mm in thickness. Sizes of about one-fifth to about five times these dimensions may also have possible uses. In some embodiments, the tip can be a separate piece that is secured to shaft by a variety of methods such as a snap mechanism, mating grooves, plastic sonic welding, etc. Alternatively, in some other embodiments, the tip can be integral or a continuation of shaft made of similar metal or materials. In some embodiments, the tip may also be constructed of materials that are both electrically non-conductive and of low thermal conductivity; such materials might comprise, for example, porcelain, ceramics, glass-ceramics, plastics, varieties of polytetrafluoroethylene, carbon, graphite, and graphite-fiberglass composites.

In some embodiments, shaft plastics, such as polytetrafluoroethylene may act as insulation about wire or electrically conductive elements. In some embodiments, the shaft may alternatively be made partially or completely of concentrically laminated or annealed-in wafer layers of materials that may include plastics, silicon, glass, glass/ceramics, ceramics carbon, graphite, graphite-fiberglass composites.

The conduit may also contain electrical control wires to aid in device operation. Partially hidden from direct view in FIGS. 4a & 4b, and located in the grooves defined by protrusions 404 are electrically conductive tissue lysing elements 405, which, when powered by an electrosurgical generator, effects lysing of tissue planes on forward motion of the device. The lysing segments may be located at the termini of conductive elements. In some embodiments, optional locations for multiple impedance sensors or multiple thermal sensors include location 410, which may be used to monitor the local post passage electrical impedance or thermal conditions that may exist near the distal tip of the shaft.

Temperature and impedance values may be tracked on a display screen or directly linked to a microprocessor capable of signaling control electronics to alter the energy delivered to the tip when preset values are approached or exceeded. Typical instrumentation paths are widely known, such as thermal sensing thermistors, and may feed to analog amplifiers which, in turn, feed analog digital converters leading to a microprocessor. In some embodiments, internal or external ultrasound measurements may also provide information which may be incorporated into a feedback circuit. In an embodiment, an optional mid and low frequency ultrasound transducer may also be activated to transmit energy to the tip and provide additional heating and may additionally improve lysing. In some embodiments, a flashing visible light source, for example, an LED, can be mounted on the tip may show through the upper skin flap to identify the location of the device.

Some embodiments may comprise a low cost, disposable, and one-time-use device. However, in some embodiments intended for multiple uses, the tip's electrically conductive tissue lysing elements be protected or coated with materials that include, but are not limited to, Silverglide™ non-stick surgical coating, platinum, palladium, gold and rhodium. Varying the amount of protective coating allows for embodiments of varying potential for obsolescence capable of either prolonging or shortening instrument life.

In some embodiments, the electrically conductive lysing element portion of the tip may arise from a plane or plate of varying shapes derived from the aforementioned materials by methods known in the manufacturing art, including but not limited to cutting, stamping, pouring, molding, filing and sanding. In some embodiments, the electrically conductive lysing element 405 may comprise an insert attached to a conductive element in the shaft or continuous with a formed conductive element coursing all or part of the shaft. In some embodiments, an electrically conductive element or wiring 411 brings RF electrosurgical energy down the shaft to electrically conductive lysing elements 405 associated in part with the recessions. In an embodiment, the electrosurgical energy via 411 is predominately electro-cutting.

In some embodiments, the electrically conductive element or wiring may be bifurcated to employ hand switching if an optional finger switch is located on handle. The electrically conductive element or wiring leading from the shaft into the handle may be bundled with other leads or energy delivering cables, wiring and the like and may exit the proximal handle as insulated general wiring to various generators (including electrosurgical), central processing units, lasers and other sources as have been described herein. In some embodiments, the plate making up lysing segments 405 may be sharpened or scalloped or made to slightly extend outwardly from the tip recessions into which the plate will fit.

Alternatively, in some embodiments, since cutting or electrical current may cause an effect at a distance without direct contact, the lysing element may be recessed into the relative recessions or grooves defined by protrusions 404 or, alternatively, may be flush with protrusions 404. In some further adjustable embodiments, locations of the electrically conductive lysing elements with respect to the protrusions may be adjusted by diminutive screws or ratchets. The plate, which in some embodiments is between 0.01 mm and 1 mm thick, can be sharpened to varying degrees on its forward facing surface. It is possible that plate sharpness may increase the efficiency with which electricity will pass from the edge cutting the target tissue. Sometimes, however, proper function even when variably dull or unsharpened may be unhampered since electrosurgical cutting current may cut beyond the electroconductive edge by a distance of over 1 mm.

In some embodiments, the electrically conductive lysing element may also exist in the shape of a simple wire of 0.01 mm to 3 mm. In some embodiments, the wire may measure between 0.1 mm and 1 mm. Such a wire may be singly or doubly insulated as was described for the plate and may have the same electrical continuities as was discussed for the planar (plate) version. In some embodiments, an electrosurgical current for the electrically conductive lysing element is of the monopolar “cutting” variety and setting and may be delivered to the tip lysing conductor in a continuous fashion or, alternatively, a pulsed fashion. The surgeon can control the presence of current by a foot pedal control of the electrosurgical generator or by button control on the shaft (forward facing button). The amount of cutting current may be modified by standard interfaces or dials on the electro surgical generator. In some embodiments, the electrosurgically energized tip current can be further pulsed at varying rates by interpolating gating circuitry at some point external to the electrosurgical generator by standard mechanisms known in the art, preferably at rates of about 1 per second to about 60 per second. For some embodiments, the electrically conductive lysing element is a monopolar tip in contact with conductive elements in the shaft leading to external surgical cable leading to an electrosurgical generator from which emanates a grounding or dispersive plate which may be placed elsewhere in contact with the patient's skin, such as the thigh.

Such circuitry may be controlled and gated/wired from the cutting current delivery system of the electro surgical generator. Acceptable electrosurgical generators may include Valley Lab Force 1 B™ with maximum P-P voltage of 2400 on “cut” with a rated load of 300 Ohms and a maximum power of 200 Watts, 35 maximum P-P voltage of 5000 on “coagulate” with a rated load of 300 Ohms, and a maximum power of 75 Watts ValleyLab Force 4 has a maximum P-P voltage of 2500 on “cut” with a rated load of 300 Ohms and a maximum power of 300 Watts, 750 kHz sinusoidal waveform output, maximum P-P voltage of 9000 on “coagulate” with a rated load of 300 Ohms and a maximum power of 120 Watts using a 750 kHz damped sinusoidal with a repetition frequency of 31 kHz. In an embodiment, the tip may also be manufactured from multilayer wafer substrates comprised of bonded conductive strips and ceramics. Suitable conductive materials include but are not limited to those already described for tip manufacture. In some embodiments, electrically non-conductive portions of the tip may comprise ceramics. In some embodiments, electrically conductive portions of the tip may comprise cermets.

In alternative embodiments, the electrically conductive lysing elements may be bifurcated or divided into even numbers at the relative recessions, insulated and energized by wiring to an even number of leads in a bipolar fashion and connected to the bipolar outlets of the aforementioned electrosurgical generators. Rings partly or completely encircling the shaft of the hand unit can be linked to a partner bipolar electrode at the tip or on the energy window. Such bipolar versions may decrease the available power necessary to electrically modify certain tissues, especially thicker tissues.

FIG. 4b is a side elevation view of the embodiment previously depicted in FIG. 4a. In the depicted embodiment, tip 401 may be made of materials that are both electrically non-conductive and of low thermal conductivity such as porcelain, epoxies, ceramics, glass-ceramics, plastics, or varieties of polytetrafluoroethylene. Alternatively, the tip may be made from metals or electroconductive materials that are completely or partially insulated. Note the relative protrusions and relative recessions are not completely visible from this viewing angle. The tip shown in this embodiment has four relative protrusions and three relative recessions and provides for a monopolar tip conductive element. In some embodiments, the relative recessions of the tip is the electrically conductive tissue lysing element 405 (usually hidden from view at most angles) which may have any geometric shape including a thin cylindrical wire; the electrically conductive lysing element can be in the shape of a plate or plane or wire and made of any metal or alloy that does not melt under operating conditions or give off toxic residua. Optimal materials may include but are not limited to steel, nickel, alloys, palladium, gold, tungsten, copper, and platinum. Metals may become oxidized thus impeding electrical flow and function. In an embodiment, the lysing element may comprise a cermet.

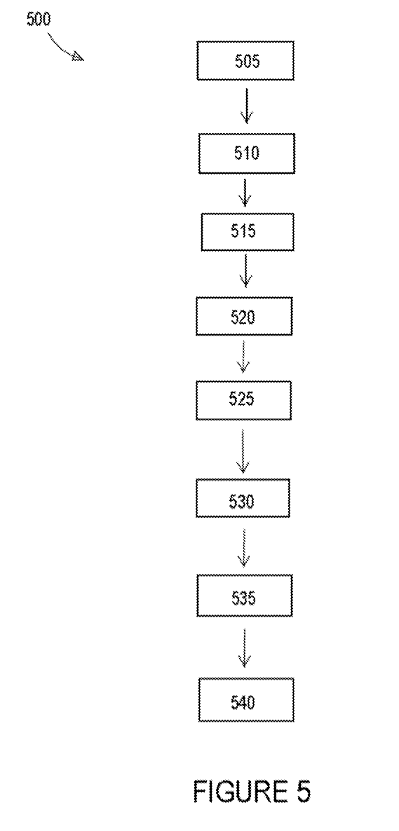

In one implementation of a method 505 according to this disclosure for incapacitating apocrine glands is shown in FIG. 5: Step 505 may comprise: having the surgical area cleaned by, for example, degreasing isopropyl alcohol (degreaser) followed by germicidal chlorhexidine scrub. Step 510 may comprise: applying a local anesthetic (such as injecting), such as about 1 cc of a 1% lidocaine+1:10,000 adrenaline, to form a 1 cm wheal/hive on the most lateral portion of the axilla. Step 515 may comprise: after allowing the local anesthetic to settle, performing a simple “stab” incision of the 1 cm wheal, for example, a #15 Bard-Parker™ Scalpel into the subcutaneous fat. This incision may be about 3 mm in length or less Step 520 may comprise: applying one or more fluids to the tissue. In some implementations, the fluid(s) may comprise water. In some implementations, the fluid(s) may comprise an ionic fluid, such as a saline solution. The fluid(s) may be applied to the tissue by, for example, injection into the stab wound(s) and may comprise a fluid that is both ionic and an anesthetic, such as a tumescent anesthesia. Some implementations may comprise applying one or more fluids that serve as an ionic fluid, an anesthetic, and an adrenaline 1n some such implementations, the fluid(s) may comprise a Klein Formula, such as about 50 cc of a Klein Formula (such as a 0.1% lidocaine+epinephrine 1:1,000,000+NaHCO3 @5 meq/L of saline). This fluid(s) may be injected into the stab wounds via, for example, a 3 mm spatula cannula and 20 cc syringe pressure, and may be fanned out to match the area to be dissected/undermined.

One or more fluids may alternatively, or additionally, be applied to the tissue by using the TDM. For example, the TDM may comprise one or more canals for delivering fluids to the tissue. In some embodiments, the canal(s) may be configured to deliver the fluid(s) adjacent to one or more of the protrusions, such as via a port located adjacent to one or more of the protrusions, for example. In some such embodiments, the canal(s) may be configured to deliver the fluid(s) in between two or more of the protrusions, such as adjacent to one or more of the lysing segments, for example. Alternatively, or additionally, the fluid(s) may be delivered elsewhere on the tip, adjacent to one or more of the energy windows, or elsewhere on the shaft of the TDM.

Step 525 may comprise: incising of the remaining portion of the wheal (such as, about 7 mm of a 1 cm wheal, for example) may then be made. This incision may be made by, for example, #15 Bard-Parker™ scalpel, into the subcutaneous fat making a total of about 1 cm in length, for example. In some implementations, Tumescent Anesthesia (TA) may be allowed to settle for about 10-30 minutes before incising of the remaining portion of the wheal (such as, about 7 mm of a 1 cm wheal, for example) may then be made. In some implementations, heat may be produced or energy may otherwise be released in the dermis or subdermis as the TDM is passed in a subdermal plane. Heat or energy from below may heat the dermis. In some implementations, heating portions of the dermis such as upper dermis or attached epidermis may be undesirable. As such, in some implementations, undesirable heating of such layers may be mitigated by a applying a cooling step antecedent and or concurrent to energy delivery with the TDM. Such steps may comprise use of a cooling mechanism such as a cooling mechanism comprising a contact cooling object such as a cooling pad or bag. Such cooling mechanism may comprise for example, a closed water bag at a temperature of less than 37° C. In some implementations, the fluid or gel may range in temperature of between 1° C. to 20° C. In some such implementations, the fluid or gel may be about 15° C. Other cooling mechanisms may comprise a dynamic cooling system wherein a cool liquid or gel is actively pumped into or though the contact cooling object. In other implementations, a thermoelectric or Peltier cooling mechanism may be applied to externally cool the skin. Step 530 may comprise: inserting TDM into the incision and fanning in about 10 strokes to cover an area of for example, about 7 cm×7 cm. Step 535 may comprise: milking the dissected area to determine if any significant bleeding or drainage is present. Step 540 may comprise: suturing the wound with, for example, 2×4-0 poliglecaprone absorbable buried interrupted stitches, followed by 1 cm of nonabsorbable running subcuticular 5-0 polypropylene stitch.

In a more general implementation of a method according to this disclosure for incapacitating apocrine glands, a first step may comprise creating an incision into a patient's skin.

A second step may comprise inserting a Tissue Dissecting and Modifying Wand into the incision and positioning the Tissue Dissecting and Modifying Wand beneath the patient's skin. The Tissue Dissecting and Modifying Wand may comprise a tip having a plurality of protrusions with lysing segments positioned between the protrusions. The Tissue Dissecting and Modifying Wand may also comprise an energy window positioned on top of the Tissue Dissecting and Modifying Wand that is configured to deliver energy to incapacitate apocrine glands.

A third step may comprise fanning out the Tissue Dissecting and Modifying Wand to define a target region within which to incapacitate apocrine glands. This step may comprise separating tissue using the lysing segment(s) to define the target region. During this step, in some implementations, the patient's skin may be placed under tension by stretching/tightening the skin at the target region during the fanning/tissue separation.

A fourth step may comprise activating the energy window and moving the energy window around within the target region to incapacitate apocrine glands. Alternatively, the energy window may be activated prior to the third step such that the step of fanning out the Tissue Dissecting and Modifying Wand to define the target region also comprises incapacitating apocrine glands within the target region.

An example of an embodiment of an apparatus according to this disclosure for incapacitating apocrine glands may comprise:

a handle;

a tip comprising a plurality of protrusions having one or more lysing segments positioned between the protrusions; and

an energy window positioned on an upper side of the apparatus, wherein the energy window comprises a thermochromic media, and wherein the thermochromic media is configured to absorb electromagnetic radiation energy and emit heat energy from the energy window.

In some embodiments as described above, the energy window may comprise a LASER that is configured to deliver energy to the thermochromic media such that the thermochromic media can then emit heat energy from the energy window.

Another example of an embodiment of an apparatus according to this disclosure for incapacitating apocrine glands may comprise:

a handle;

a tip comprising a plurality of protrusions having one or more lysing segments positioned between the protrusions; and

an energy window positioned on an upper side of the apparatus, wherein the energy window comprises an target-tissue-impedance-matched-microwave-based energy window.

It will be understood by those having skill in the art that changes may be made to the details of the above-described embodiments without departing from the underlying principles presented herein. For example, any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

1. A method for incapacitating apocrine glands, the method comprising the steps of:
creating an incision into a patient's skin;
inserting a tissue dissecting and modifying wand into the incision and positioning the tissue dissecting and modifying wand beneath the patient's skin; wherein the tissue dissecting and modifying wand comprises:
a tip comprising a plurality of protrusions; and
at least one lysing segment positioned between each of the protrusions;
defining a target region for incapacitating apocrine glands; and
using the tissue dissecting and modifying wand to at least partially incapacitate the apocrine glands within the target region.

2. The method of claim 1, wherein the tissue dissecting and modifying wand further comprises an energy window configured to deliver energy to incapacitate apocrine glands.

3. The method of claim 2, wherein the energy window is positioned on an upper surface of the tissue dissecting and modifying wand.

4. The method of claim 2, wherein the energy window comprises a plurality of energy delivery regions within which energy is delivered and a plurality of interspersed regions within which no energy is delivered.

5. The method of claim 2, wherein the energy window comprises a thermochromic media, and wherein the thermochromic media is configured to absorb electromagnetic radiation energy and emit heat energy from the energy window.

6. The method of claim 2, wherein the energy window comprises a target-tissue-impedance-matched-microwave based energy window.

7. The method of claim 2, wherein the energy window comprises at least one of radiofrequency, microwave, intense pulsed light, LASER, thermal, and ultrasonic energy.

8. The method of claim 1, wherein the step of defining a target region for incapacitating apocrine glands comprises separating tissue into at least two tissue planes using the at least one lysing segment.

9. The method of claim 8, wherein the step of defining a target region for incapacitating apocrine glands comprises tightening a patient's skin at the target region.

10. The method of claim 1, wherein the tissue dissecting and modifying wand is used to at least partially incapacitate the apocrine glands within the target region while the tissue dissecting and modifying wand is used to define the target region.

11. The method of claim 1, wherein the step of defining a target region for incapacitating apocrine glands comprises fanning out the tissue dissecting and modifying wand to define the target region.

12. The method of claim 1, wherein the target region at least partially comprises the patient's underarm region.

13. The method of claim 12, wherein the step of using the tissue dissecting and modifying wand to at least partially incapacitate the apocrine glands within the target region comprises using the tissue dissecting and modifying wand to incapacitate at least substantially all of the apocrine glands within the target region.

14. A method for incapacitating apocrine glands, the method comprising the steps of:
creating an incision into a patient's skin;
inserting a tissue dissecting and modifying wand into the incision and positioning the tissue dissecting and modifying wand beneath the patient's skin; wherein the tissue dissecting and modifying wand comprises:
a tip comprising a plurality of protrusions;
at least one electrically conductive lysing segment positioned between each of the protrusions and configured to separate tissue into at least two tissue planes; and
an energy window configured to deliver energy to incapacitate apocrine glands, wherein the energy window is positioned adjacent the protrusions of the tissue dissecting and modifying wand;
fanning out the tissue dissecting and modifying wand to define a target region for incapacitating apocrine glands;
separating tissue into at least two tissue planes using the at least one lysing segment;
using the energy window of the tissue dissecting and modifying wand to incapacitate at least substantially all of the apocrine glands within the target region.

15. The method of claim 14, wherein the tissue dissecting and modifying wand further comprises:
a handle; and
a shaft positioned in between the tip and the handle.

16. The method of claim 15, wherein the energy window is positioned on an upper surface of the shaft.

17. The method of claim 14, wherein the target region at least partially comprises the patient's underarm region.

18. A method for incapacitating apocrine glands, the method comprising the steps of:
inserting a tissue dissecting and modifying wand into a patient's skin below the dermis layer of the skin, wherein the tissue dissecting and modifying wand comprises:
a tip comprising a plurality of protrusions;
at least one lysing segment positioned between each of the protrusions; and
an energy window positioned adjacent to the tip;
positioning the tissue dissecting and modifying wand into the patient's skin below the dermis layer of the skin such that the energy window is facing the dermis layer of the skin;
defining a target region for incapacitating apocrine glands by moving the tissue dissecting and modifying wand around within the target region to separate the patient's tissue within the target region into separate tissue planes, wherein one of the tissue planes comprises the dermis layer;
activating the energy window; and
using the energy window to at least partially incapacitate the apocrine glands within the target region.

19. The method of claim 18, wherein the step of defining a target region for incapacitating apocrine glands comprises fanning out the tissue dissecting and modifying wand below the dermis layer to define the target region.

20. The method of claim 18, wherein the step of using the energy window to at least partially incapacitate the apocrine glands within the target region is performed at least partially at the same time as the step of defining the target region.

21. The method of claim 18, wherein the step of using the energy window to at least partially incapacitate the apocrine glands within the target region is performed subsequent to the step of defining the target region.

22. The method of claim 18, wherein the target region at least partially comprises the patient's underarm region.

23. The method of claim 18, wherein the step of using the energy window to at least partially incapacitate the apocrine glands within the target region comprises using the energy window to incapacitate at least substantially all of the apocrine glands within the target region.
